(19) United States
(12) Patent Application Publication
(10) Pub. No.: US 2008/0033426 A1
(43) Pub. Date: Feb. 7, 2008

(54) Catheter system and method of use thereof

(76) Inventor: Charles H. Machell, San Antonio, TX (US)

Correspondence Address: Daniel D. Chapman;Jackson Walker, LLP, Suite 2400, 112 E. Pecan, San Antonio, TX, 78205, US

(21) Appl. No.: 11/881,317

(22) Filed: Jul. 26, 2007

Related U.S. Application Data
(60) Provisional application No. 60/833,649, filed on Jul. 27, 2006.

(51) Int. Cl. A61B 18/18

(52) U.S. Cl. 606/41

(57) ABSTRACT

A catheter system including a catheter having a shaft ablation electrode. An embodiment discloses a switch in a catheter having both a tip and a shaft ablation electrode for switching the delivery or RF energy between the two ablation electrodes. Further, a method of using a catheter having a shaft ablation electrode for treating heart disease and disorders. The method includes use of the shaft ablation electrode for ablation of tissue on a ring or ridge shaped structure.

This application claims priority to and incorporates herein by reference U.S. Provisional Patent Application Ser. No. 60/833,649, filed Jul. 27, 2006.

### FIELD OF THE INVENTION

Catheter systems, including catheter systems for use in diagnosis and treatment, as by RF energy, of organs in the human body, including the heart.

### BACKGROUND OF THE INVENTION

Ablation is a medical term that refers to any procedure performed to destroy diseased or damaged tissue in the human body. Catheter ablation is a technique in which a thin tube or catheter is inserted percutaneously and manipulated through the blood vessels to the site of origin of an arrhythmia. Target tissue is heated by applying energy through the catheter, usually using radiofrequency current, to destroy cardiac tissue responsible for the genesis of arrhythmias.

Radiofrequency is a widely used and effective form of energy applied during catheter ablation. Alternating current, typically between 300-750 kHz, is applied through the catheter causing resistive heating of the tissues in contact with the RF electrode of the catheter. The power of the RF pulses is typically controlled by the catheter tip temperature and system impedance. Typical ablation catheters have a 4 mm distal (tip) electrode and create lesions approximately 4-6 mm in diameter and 2-3 mm deep. Larger electrodes (8-10 mm) produce larger lesions.

Over the years, the electrophysiological effects of radiofrequency catheter ablation (RFCA) have proved to be beneficial and are now the first line of treatment of certain heart conditions, such as supraventricular tachyarrythmias, Wolff-Parkinson-White syndrome, and atrial tachyarrhythmias including atrial fibrillation and right and left-sided atrial flutters.

Among the heart conditions that have been successfully treated with RFCA are supraventricular tachycardia and Wolff-Parkinson-White syndrome, conditions that affect both the tricuspid annulus and the mitral annulus. Both of these are ring structures with a perimeter having a thickness typically between 2 and 4 mm. In treating conditions of the mitral and/or tricuspid annulus with the RF catheter described above, it is often difficult to maintain a distal ablation electrode adjacent the ring structure as often a slight amount of pressure against the ring will cause the tip of the catheter to slip off the target tissue. As a result, a significant amount of time required for RF catheterization of these and other tissue structures with a peak, ridge or ring shape is consumed with proper location of the catheter tip adjacent the target area. When the location of the target area is close to or at a peak, ridge or ring, the tip electrode must be carefully placed on the target site and this careful placement is time consuming. Areas, such as areas within the heart, that are difficult to maintain tip electrode contact may be described as “non-planar” areas and, more particularly, areas that have a radius of curvature of less than about 2 times the radius of curvature of the tip of the catheter. Typical catheters are between 5 and 8 french (in diameter) and thus the range of radii of curvature of such non-planar areas would be up to about 16 french. Among the structures that might typically be found to be in this range are: tricuspid annulus, mitral annulus, and pulmonary vein orifices.

Good electrode contact with target tissues is essential to perform definitive lesions. Unstable catheter positions and changes on the lesion border zone may explain why arrhythmias may recur after apparently successful RF ablations.

Over the years, and with sufficient practice, heart surgeons have become adept at tip electrode placement in such non-planar areas. Nonetheless, a device and method of using a device which would make RF ablation a faster and simpler procedure, especially in the non-planar areas described, would be beneficial.

### OBJECTS OF THE INVENTION

It is an object of the present invention to provide a device that will make it easier and quicker to apply RF energy to a ring, peak, ridge or other non-planar tissue structure.

### SUMMARY OF THE INVENTION

This and other objects are provided in a catheter system comprising a radio frequency generator engaged with a catheter having a catheter shaft, a handle, and at least one shaft ablation electrode mounted on the catheter shaft proximal to the tip of the catheter shaft.

This and other objects are provided in the catheter system set forth in the paragraph above, which further includes a tip ablation electrode mounted to the tip of the catheter shaft.

This and other objects are provided in the catheter system set forth in the two paragraphs above, further including a switch, mountable in a number of places, including either on the handle or separate from the handle, which switch is capable of switching between a tip electrode and a shaft mounted electrode for the application of RF energy to either of the tip electrode or shaft electrode.

This and other objects are provided in the catheter system and method of use of the catheter system described in the paragraphs above, which is capable of selectively choosing, through the switch, including a lockable switch, one of a tip or shaft electrode for application of RF energy to a target issue adjacent the selected electrode.

This and other objects are provided in the catheter system and method of use of the catheter system described in the paragraphs above, wherein the method of use comprises the steps of percutaneously introducing the catheter system through a blood vessel and selecting either the shaft ablation electrode or the tip ablation electrode, in positioning the selected electrode on tissue of the organ and applying RF energy to the selected ablation electrode.

This and other objects are provided in the catheter system having only four electrodes, a tip and a ring ablation electrode and a pair of recording electrodes, to enhance flexibility of the catheter shaft and tip area and therefore increase maneuverability and ease of placement on a ring structure.

### BRIEF DESCRIPTION OF THE DRAWINGS

FIG. 1 illustrates a catheter with a handle and cable attached thereto in elevational view.

FIGS. 2 and 3 are side elevational views of the distal end of two preferred embodiments of applicant's present invention.

FIG. 4 is a partial view in perspective of part of the handle and catheter cable of applicant's present invention.

FIG. 5 is an elevational view of an RF generator, and diagnostic and catheter cables for use with applicant's present invention.

FIG. 6 is an illustration of the physical equipment comprising part of applicant's invention, including the use of a switch box.

FIGS. 7A-7C illustrate applicant's method of use of the catheter system.

FIG. 8 is a cross-sectional view of the catheter taken along the line A-A′ in FIG. 1.

FIG. 9 is longitudinal cutaway view of the catheter according to applicant's present invention.

FIG. 10 is a cross-sectional view of the catheter taken along the line B-B′ in FIG. 2.

FIG. 11 is a functional block diagram for the electronics according to an embodiment of the invention.

FIG. 12 is a circuit diagram of the switching mechanism of according to another embodiment of the invention.

FIG. 13 is a drawing of a switching mechanism according to one embodiment of the invention.

FIG. 14 is a drawing of a switching mechanism according to another embodiment of the invention.

### DETAILED DESCRIPTION OF THE PREFERRED EMBODIMENTS

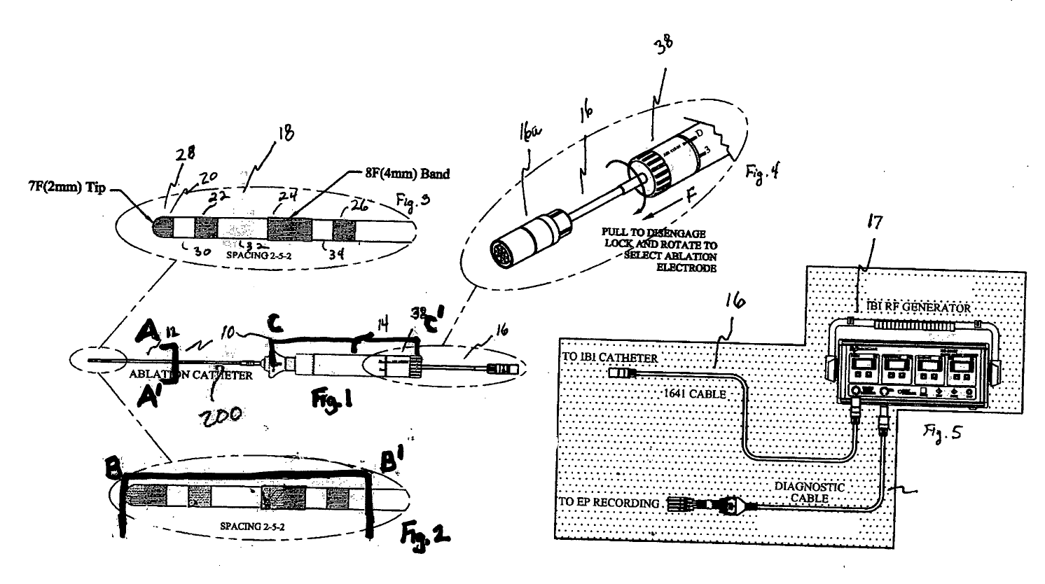

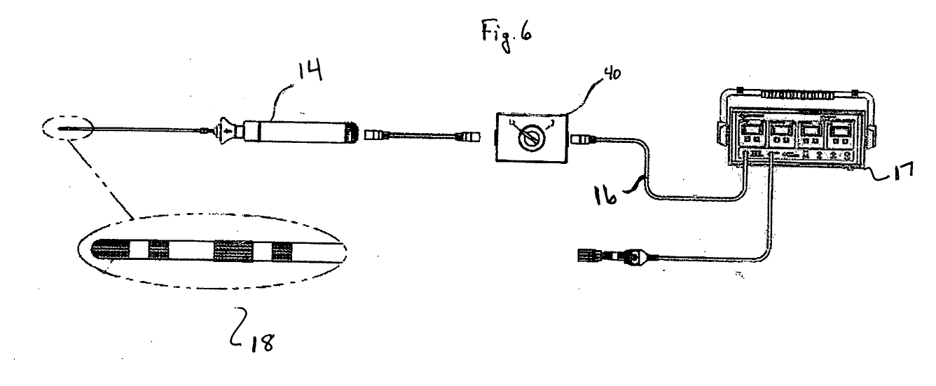

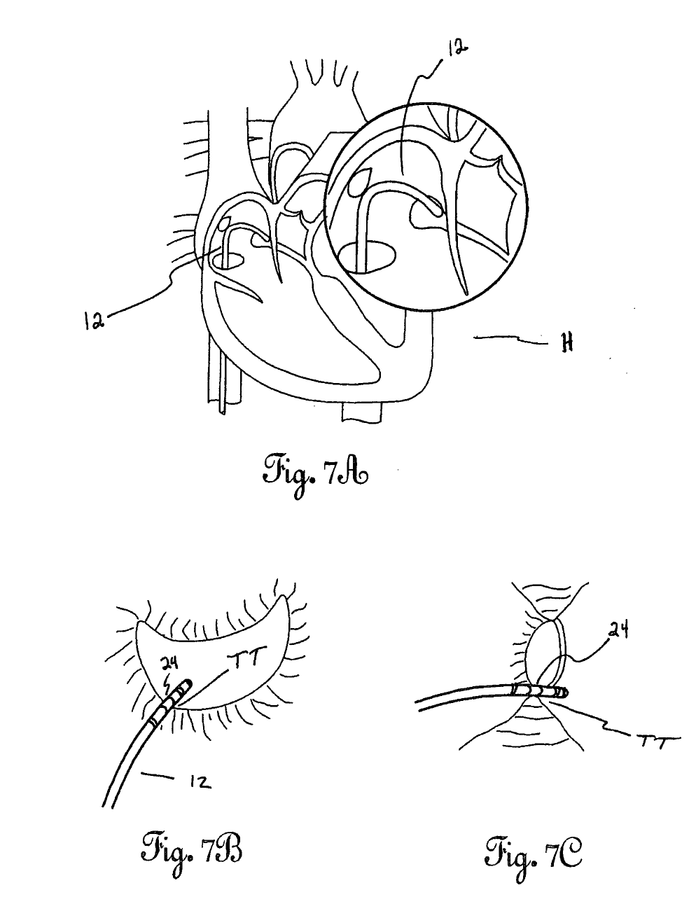

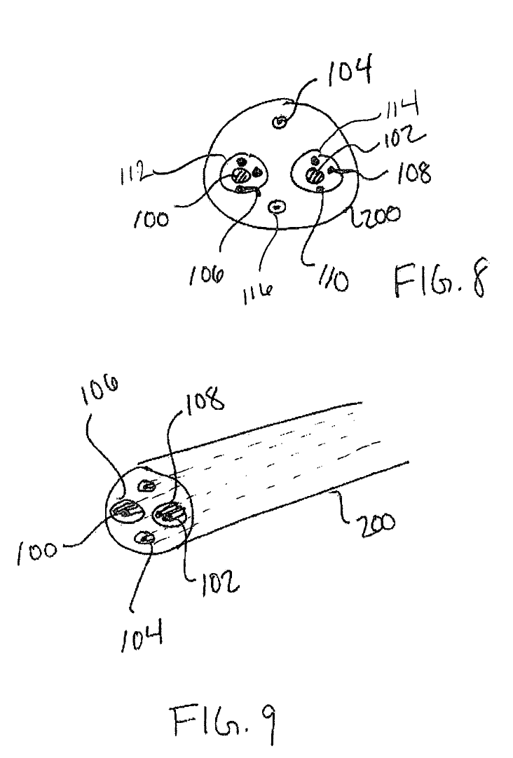

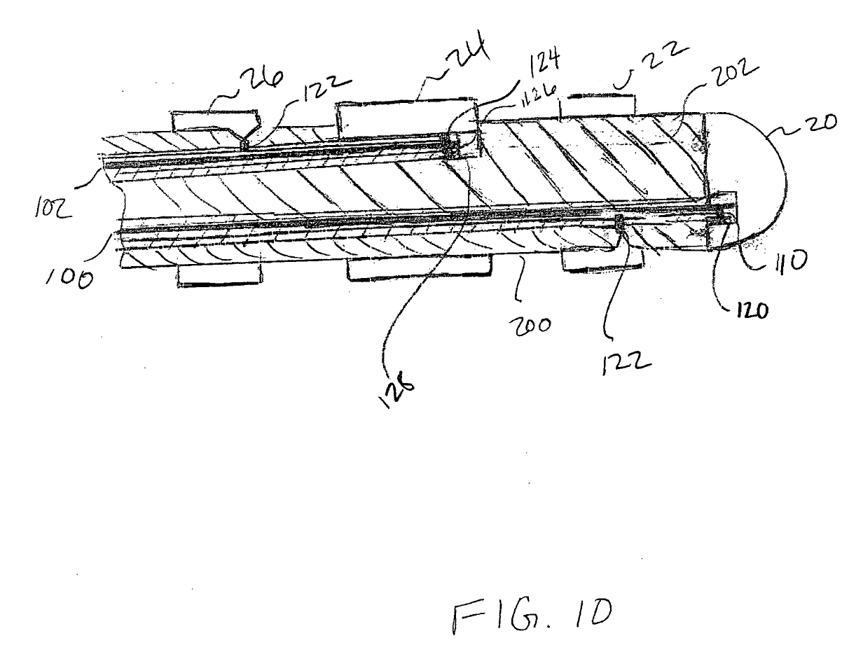

FIG. 1 illustrates applicant's catheter 10, the catheter including a cylindrical catheter shaft 12 engaged with a catheter handle 14. A catheter cable 16 connects the catheter to a radio frequency RF generator 17 (see FIG. 5) for electronic engagement of the catheter with the RF generator.

The catheter shaft 12 includes a shell 200, fabricated from a nylon, urethane or other plastic, bio-compatible material and a polyamide layer 202 forming electrode lumens 112 and control lumens 114, therein. The electrode lumens and the control lumens are essentially long hollow tubes that traverse the length of the catheter shaft 12.

Turning now to FIG. 2, it is seen that the external portion of applicant's catheter shaft 12 includes a distal end 18 typically comprised of a multiplicity of electrodes (preferably four), here electrodes 20, 22, 24, and 26. Distal end 18 includes a tip 28. As set forth in the Figures, tip 28 may be comprised of an electrode, here distal or tip electrode 20. Electrodes may be defined in terms of their placement either on the shaft or at the tip, a distal or tip electrode 20 being defined as one which comprises at least part of tip 28. Non-tip or shaft electrodes, here electrodes 22, 24, and 26, for example, are cylindrical and typically have a shaft portion of the catheter exposed distal to their locations.

Electrodes 20, 22, 24, and 26 are typically separated by inter-electrode areas 30, 32, and 34. As is known in the art, electrodes are typically comprised of a metallic or other conductive material (copper, platinum or gold, for example). The electrodes may serve the functions of recording for diagnostic purposes or ablation for therapeutic purposes. In the embodiment illustrated in FIGS. 2 and 3, electrodes 20 and 24 are ablation electrodes and electrodes 22 and 26 are recording electrodes.

In FIG. 2, the size of the distal or tip electrode 20 is about 4 millimeters in length and about 7 F diameter, on a 7 F shaft. In the embodiment illustrated in FIG. 3, the tip electrode 20 is about 2 mm in length, about 7 F in diameter on a 7 F shaft.

In both embodiments illustrated (FIGS. 2 and 3), the spacing is 2-5-2, that is, 2 mm, 5 mm, and 2 mm, representing the distance between adjacent electrodes. In an alternate preferred embodiment, spacing is 2-2-2. With the 2-2-2 spacing and single 4 mm shaft electrodes, with the remaining three electrodes being 22 mm, there is a 16 mm distance between the end of the tip and the first recording electrode (26). Using the 2-5-2 spacing, 4 mm tip, 4 mm shaft electrode 24 and 22 mm recording electrodes 22 and 26, the distance between the tip and the first recording electrode 26 is 21 mm. Applicant's preferred embodiment has four electrodes, a tip and three shaft electrodes, and the distance between the end of the tip and the first recording electrode 26 is in a range of 16 to 21 mm. The shorter the distance, the more flexibility typically provided, which may be beneficial when applicant's novel catheter system is used in the method described herein.

Turning now to FIGS. 8-10, the internal portion of applicant's catheter shaft will be described. As previously mentioned, catheter shaft 12 includes control lumens 114 and electrode lumens 112. Electrode lumens have several wires disposed therein, namely thermocouple wires [not shown], EKG sensing wires 106 and 108, and ablation wires 100 and 102. As can be seen, each electrode lumen has only one ablation wire 100/102. As one skilled in the art will appreciate, ablation wires 100/102 are preferably formed from coaxial cable suitable to transmit radio frequency signals at the desired level. Thermocouple wires {not shown], are preferably made from copper and constantan. EKG sensing wires are fabricated from a material having a low work function, e.g. copper and the like, and are surrounded by an insulative sheath.

The control lumens 114 in the catheter shaft carry the control wires 104. Control wires 104 are connected to the [handset] and the distal tip of the catheter shaft 12 to control the placement of the ablation electrodes 20 and 24. Control wires are fabricated from, for example, stainless steal, but other wire materials are possible and within the scope of this disclosure. In the exemplary embodiment of the invention, there are two control lumens 114 carrying two control wires 104, however, the control of electrode tips using control wires is not novel, and apparatuses that use one control wire, three control wires, etc., are within the prior art and the scope of this invention.

The catheter shaft terminates into the catheter tip 18. A cross-sectional view of the catheter tip is described in FIG. 10. As previously mentioned, the catheter tip includes several electrodes 20, 22, 24, and 26, and the connection between the electrodes 20, 22, 24, and 26 and the thermocouple wires, the EKG sensing wires and the ablation wires in lumens 112 will now be described. A first lumen 112 terminates at approximately the location of the distal tip electrode 20. Distal tip electrode 20 has a recessed portion therein containing a ceramic bushing 110. The ablation electrode wire 100 extends beyond the ceramic bushing to contact distal tip electrode 20. However, the thermocouple terminal 120 [the joining portion of the two different metal conductors] is formed at an opening between the ceramic bushing and the electrode tip. In this way, the wires of the thermocouple are isolated from the ablation electrode wire 100, and any crosstalk between the ablation electrode wire 100 and the thermocouple is minimized. EKG sensing wires 106 are also disposed in the first lumen 112. The EKG wire 106 is connected to the electrode 22, and terminates at a conductive bushing 122, which may or may not be partially covered in an insulative material. Conductive bushing 122 extends from lumen 112 into the polyamide layer. As is showing in FIG. 10, the a portion of the electrode 22 is adapted to pierce the polyamide layer and contact the conductive bushing 122. In this way, electrode is wired through the catheter shaft to the RF generating unit.

A second lumen 114 contains the wiring for electrodes 24 and 26, and at the terminal end of the lumen 114, a second ceramic bushing 124 and a conductive terminal ring 126 are disposed. Since lumen 114 does not extend the length of the catheter tip, the conductive terminal ring 126 is adapted to electrically connect the second ablation electrode 24 and the second ablation electrode wire 102. To accomplish this, the conductive terminal ring 126 is in communication with the ablation electrode wire 102, the conductive terminal ring 126 extends from the second lumen 114 through a portion of the polyamide layer, and the electrode 24 is adapted to pierce the polyamide layer to contact the conductive terminal ring 126.

The thermocouple [not shown], ceramic bushing 120 and EKG sensing wires are also disposed in the second lumen 114. Much like ceramic bushing 120, ceramic bushing 124 electrically isolates the thermocouple terminal and is arranged at an opening between the ceramic bushing 124 and the conductive terminal ring 126. The EKG sensing wire 108 is also disposed within the second lumen 114. The EKG sensing wire 108 terminates at a second conductive bushing 122 that extends into the polyamide layer. The electrode 26 is adapted to also pierce the polyamide layer, and contact the EKG sensing wire 108.

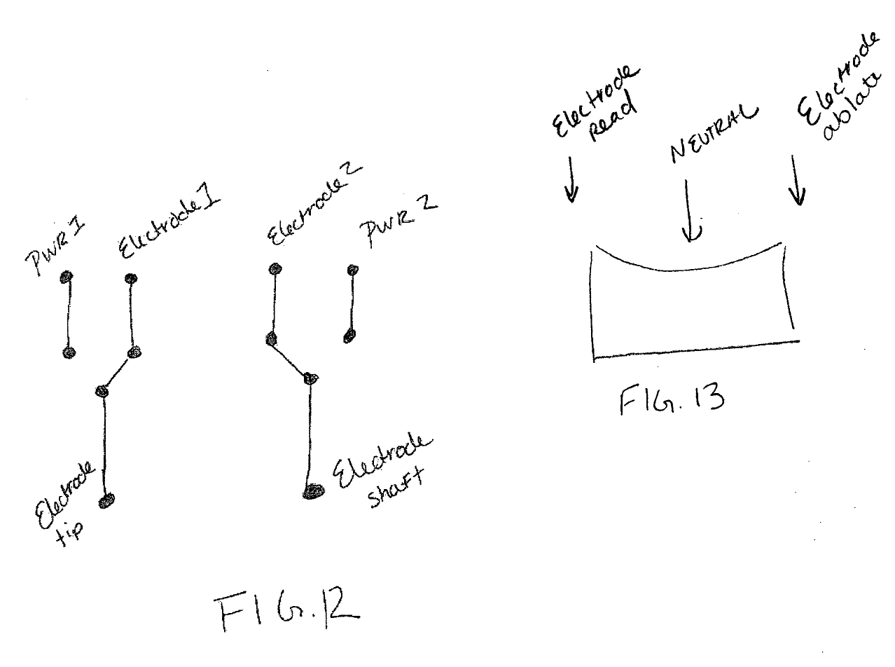

As is show in FIGS. 8 and 9, the ablation electrode wires, the thermocouple wires and the EKG sensing wires run the length of the shaft in lumens 112 and 114.

As previously mentioned, the catheter shaft described above is connected to a handle 14. As shown in FIG. 15, handle 14 provides a control mechanism for engaging with the wires in the control lumens and an electronic switching mechanism. The control mechanism for engaging with the wires in the control lumens is well known in the prior art, and includes a shaft 204 and actuator 206, which are slideably connected, and fabricated from a hard moldable material, e.g., plastics or the like. The shaft 204 is sized so as to closely fit around the bottom portion of the actuator 206 while still allowing the actuator to move in and out of the shaft 204. A screw means 208 is provided at the junction between the shaft 204 and the actuator 206, and allows an operator to restrict the amount of slideable movement. Inside the handle 14, an anchor 210 hold a terminal end of control wires 104, the control wires extending from the anchor, through the shaft and actuator into two separate lumens of the catheter shaft 12.

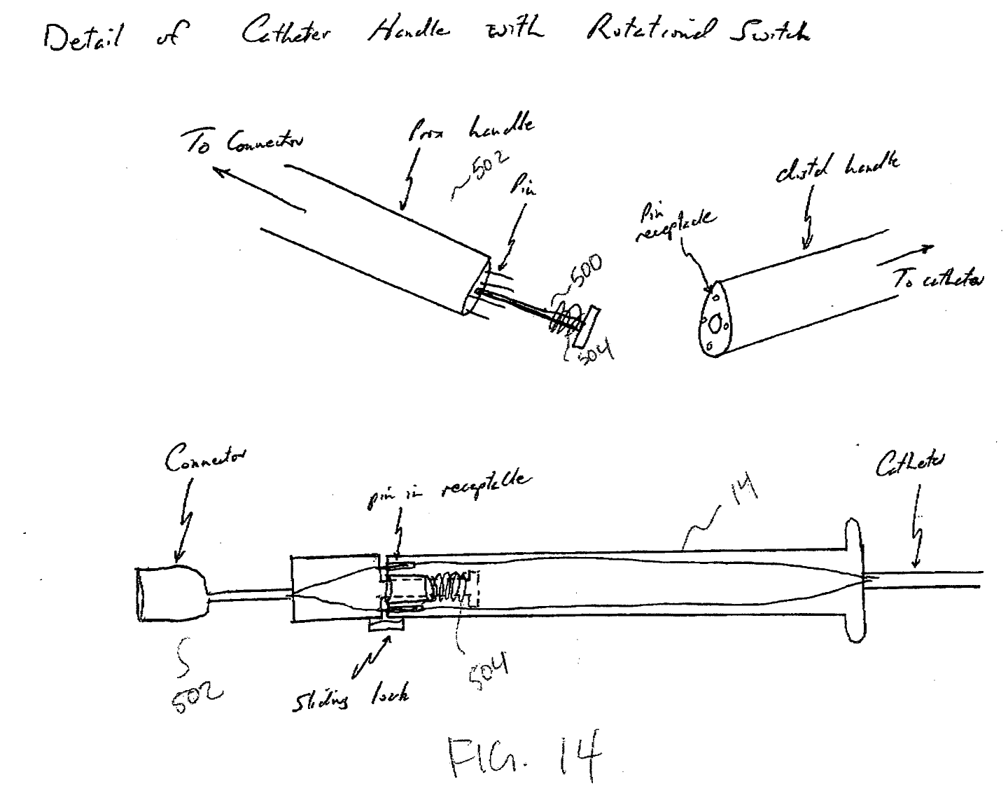

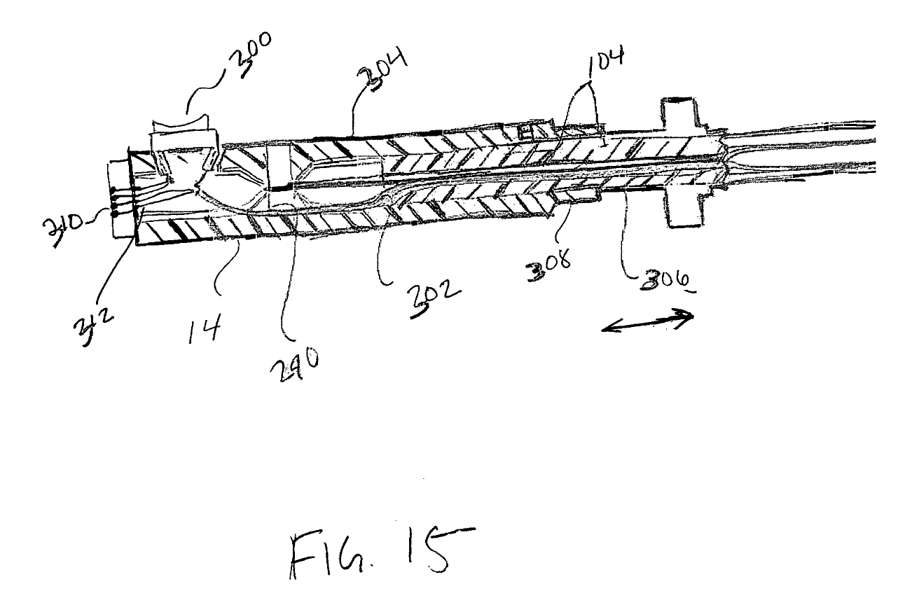

Now the operation of the device according to an embodiment of the invention will be shown with reference to FIGS. 11-13.

With reference to FIGS. 2 and 3, one embodiment of applicant's novel invention provides preferably one shaft electrode (that is, an electrode proximal to tip 20), as electrode 24, which is an ablation electrode. In a preferred embodiment, there is only one shaft electrode that is an ablation electrode and a tip electrode that is an ablation electrode. In an embodiment of applicant's present invention, an ablation electrode is also provided as tip electrode 20. In the embodiments of applicant's invention set forth in FIGS. 2 and 3, it is seen that both a tip and a shaft electrode, here 20 and 24, are engaged through the handle and a switch 38, 40 to the RF generator 17 for applying RF energy to catheterized tissue.

The electrodes 22 and 26 are, in the embodiments illustrated in FIGS. 2 and 3, recording electrodes for diagnostic purposes from which local tissue electrograms and other measurements may be obtained.

In one embodiment of applicant's present invention, one or more shaft mounted ablation electrodes 24 are provided along with an ablation tip electrode 20, where the shaft mounted ablation electrode may be 8 F in diameter mounted on a 7 F shaft. It is believed that providing a shaft mounted RF electrode with a larger diameter than the shaft, here a 1 F difference between the shaft diameter and the diameter of the shaft mounted RF electrode, will help achieve a more effective RF lesion.

In the alternative, while not pictured, the shaft mounted RF electrode(s) may have the same diameter as the shaft, for example, 7 F diameter or other depending on the catheter diameter.

One of the objectives of applicant's present invention is to provide for all of the advantages of a catheter having an RF tip electrode with one that could effectively treat non-planar areas, ridges, peaks, annuli or rings, such as structures including, for example, the mitral annulus and the tricuspid annulus.

In a preferred embodiment of applicant's present invention, a switch may be provided for switching between the application of RF energy to tip electrode or non-tip electrode (shaft electrode) 24. That is, in a single catheter, the operator is provided with a switch to selectively switch RF energy delivery between tip electrode 20 and shaft electrode 24 and thus may have the advantages of treating a non-planar or planar areas, through the use of shaft mounted ablation electrode 24 or tip electrode 20.

Applicant provides two locations for switches 30, 40, here in handle switch 38, wherein the switch is incorporated into the handle and removed switch 40 (see FIG. 6) when switch is not located on handle 20. Two forms of a switch 38, 40, here handle switch 38, or removed switch 40, are provided. Both switches 38, 40 are surgeon controlled, typically manually, and achieve the function of directing RF energy selectively either to a tip electrode or a shaft mounted electrode, but switch 38 is mounted on and part of handle 14 and switch 40 is separate from handle 14. Switch 38 has a number of advantages, including ease of access. Handle 14 is grasped by the surgeon during an ablation procedure and mounted switch 38 on the handle provides easy access to the surgeon. Non-handle switch 40 may require a second person to operate, being removed as it is, from the handle. Both switches 38 and 40 may be manually operated and serve to selectively deliver RF energy to either a tip or shaft mounted catheter electrode.

Turning to FIG. 4, it is seen that switch 38 is engaged to a distal end of cable 16, cable 16 having a proximal end including connector 16a for connection to RF generator 17 in a manner known in the art. Switch 38 may be a rotary two-position switch, including a lockout feature in which an axial force F may be applied against bias, in the direction indicated by the arrow to disengage the switch, which in such disengaged position may be rotated between position D for distal tip or position designated 3 for shaft mounted ablation electrode.

FIG. 6 illustrates a switch box (without a lockout feature) separated from the handle and typically located between the RF generator and the handle for selectively switching energy between the tip or shaft mounted RF electrode. In an alternative embodiment, the RF generator may include a switch built in to the RF generator or a floor mounted foot operated “pedal” switch. Other types of switches, either mounted on the handle or apart therefrom, may be rocker switches, rotary switches, toggle switches, slide switches, or other types of switches.

The method and apparatus of the present invention are intended for the delivery of radio frequency energy to a target location within an interior chamber of a patient's heart. Radio frequency ablation involves the application of radiofrequency energy, typically at a frequency range from about 250 to 1000 kHz, but usually in the range of 400 to 500 kHz, at a time and temperature that induces necrosis in the tissue abated. Typically, the tissue temperature is above around 45° C., usually above about 60° C., and preferably maintained below 95° C. For such temperatures the radio frequency energy will be applied for time periods in the range of 30 to 60 seconds, but may be applied at times as low as seconds, or as high as 90 seconds.

In order to deliver the radio-frequency energy to the desired target location within the heart, the catheter system of the invention, having a suitable electrodes near its distal end, will be percutaneously introduced, typically through the femoral vein or artery in the patient's groin. The distal tip and is then manipulated through conventional means, typically through a guiding catheter, until it reaches the interior of the heart. The electrode tip of the catheter will then be further manipulated until it reaches the area to be ablated. Radio-frequency energy is then applied to the target location in a method described herein and preferably using the novel catheter system of the invention.

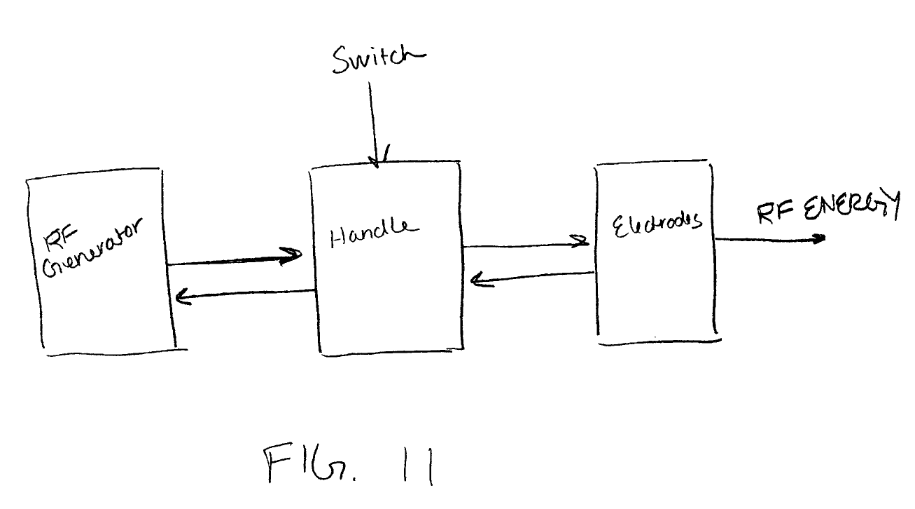

FIGS. 7A-7C illustrate a method of use of applicant's novel catheter. A method of use would typically include the following steps:

a. percutaneously introducing the catheter having both shaft and tip electrodes through a blood vessel to an organ, such as the heart H;

b. identifying a target tissue for ablation;

c. determining the suitability of the target tissue for ablation with either a tip electrode or a shaft electrode;

d. positioning the selected electrode against a target area;

e. selecting the desired electrode and applying RF energy to the target site.

The determining step may be achieved by simply identifying the structure itself, with the surgeon's general knowledge of the typical size of such a structure. For example, the mitral or tricuspid annuli would typically be within the size range suitable for use with a shaft mounted ablation electrode and would be identified as such. In other cases, the practitioner would visually examine the tissue structure using fluoroscopy and/or examine electrograms recorded from the catheter and through either manual placement of the tip electrode, and/or shaft electrode, probe to determine its effectiveness and ease in positioning or the placement would determine by such simple experimentation in a patient which electrode the surgeon would choose. Upon choosing the appropriate electrode, the practitioner would switch to the appropriate electrode and proceed with the ablation. The shaft electrode is typically placed in non-parallel relation to a structure with an elongated ridge or ring (annulus) shape, and most preferably perpendicular thereto (see FIG. 2).

Although the invention has been described with reference to specific embodiments, this description is not meant to be construed in a limited sense. Various modifications of the disclosed embodiments, as well as alternative embodiments of the inventions, will become apparent to persons skilled in the art upon the reference to the description of the invention. It is, therefore, contemplated that the appended claims will cover such modifications that fall within the scope of the invention.

1. A catheter system comprising:
an RF generator:
a catheter shaft having a shaft diameter, a tip, a distal end, and a proximal end;
a handle for attachment to the proximal end of the catheter shaft; and
at least one shaft ablation electrode mounted on the catheter shaft proximal to the tip of the catheter shaft.

2. The catheter system of claim 1 further comprising a tip ablation electrode mounted at the tip of the catheter shaft.

3. The catheter system of claim 2, wherein the handle includes a manual switch for providing RF energy from the RF generator to either of the shaft ablation catheter or the tip ablation catheter.

4. The catheter system of claim 3, wherein the manual switch is one of the following:
rocker, rotary, toggle, slide or other.

5. The catheter system of claim 3, wherein the handle has a proximal end and a distal end, wherein the manual switch is located nearer the proximal end thereof.

6. The catheter system of claim 2, further including a manual switch for placement between the source of RF energy and the catheter handle to selectively deliver RF energy to one of the tip ablation electrode or the shaft ablation electrode.

7. The catheter system of claim 6, wherein the switch is adapted to releasably lock in a selected position.

8. The catheter system of claim 1, further including at least two recording electrodes.

9. The catheter system of claim 2, further including at least two recording electrodes.

10. The catheter system of claim 9, wherein at least two recording electrodes include only two recording electrodes.

11. The catheter system of claim 10, wherein the two recording electrodes include one located between the tip ablation catheter and the shaft ablation catheter and one located proximal to the shaft ablation electrode.

12. The catheter system of claim 11, wherein the spacing between the electrodes is 2-5-2.

13. The catheter system of claim 11, wherein the spacing between the electrodes is 2-2-2.

14. The catheter system of claim 1, wherein the length of the distal end of the shaft is between 1 and 3½ centimeters.

15. The catheter system of claim 2, wherein the length of the distal end is between 1 and 3½ centimeters.

16. The catheter system of claim 1, wherein the diameter of the shaft is between 5 and 8 french.

17. The catheter system of claim 2, wherein the diameter of the shaft is between 5 and 8 french.

18. The catheter system of claim 1, wherein the shaft ablation electrode has a diameter greater than the shaft.

19. The catheter system of claim 18, wherein the difference between the shaft ablation electrode and the shaft of the catheter is in the range of 1 french to 3 french.

20. The catheter system of claim 2, further including a switch for selectively energizing either the tip ablation electrode or the shaft ablation electrode.

21. The catheter system of claim 2, wherein the shaft ablation electrode has a diameter greater than the shaft.

22. The catheter system of claim 21, wherein the difference between the shaft ablation electrode and the shaft of the catheter is in the range of 1 french to 3 french.

23. A method for cauterizing tissue within a heart chamber, the method comprising the steps of:
providing a catheter system comprising: an RF generator; a catheter shaft having a shaft diameter, a tip, a distal end, a controlled portion and a proximal end; a handle for attachment to the proximal end of the catheter shaft; and one shaft ablation electrode mounted on the catheter shaft proximal to the tip of the catheter shaft;
percutaneously introducing the catheter system through a blood vessel to the heart chamber;
identifying a target tissue that is not appropriate for locating thereupon a tip electrode but is appropriate for locating the shaft electrode thereupon;
positioning the shaft ablation electrode on the identified tissue; and
applying RF energy to the shaft ablation electrode.

24. A method for cauterizing tissue within a heart chamber, the method comprising the steps of:
providing a catheter system comprising: an RF generator; a catheter shaft having a shaft diameter, a tip, a distal end, a controlled portion and a proximal end; a handle for attachment to the proximal end of the catheter shaft; one shaft ablation electrode mounted on the catheter shaft proximal to the tip of the catheter shaft; and a tip ablation electrode mounted on the tip of the catheter shaft;
percutaneously introducing the catheter system through a blood vessel to the heart chamber;
identifying target tissue and determining which of the shaft electrode or the tip electrode is more appropriate;
selecting one of the shaft ablation electrode or the tip ablation electrode;
positioning the selected electrode on the target tissue; and
applying RF energy to the selected ablation electrode.

25. The method of claim 22, wherein the selected electrode is the shaft electrode and the target tissue includes tissue on one of the following: mitral annulus, tricuspid annulus, pulmonary vein orifice, atrial or ventricular myocardium.
